(19) United States
(12) Patent Application Publication
(10) Pub. No.: US 2008/0262064 A1
(43) Pub. Date: Oct. 23, 2008

(54) Novel Compounds For The Treatment Of GI Disorders 682

(75) Inventors: Udo Bauer, Molndal (SE); Wayne Brailsford, Molndal (SE); Leifeng Cheng, Molndal (SE); Maria Jonforsen, Molndal (SE); Florian Raubacher, Molndal (SE); Peter Schell, Molndal (SE); Tor Svensson, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/104,464

(22) Filed: Apr. 17, 2008

Related U.S. Application Data
(60) Provisional application No. 60/912,526, filed on Apr. 18, 2007.
(60) Provisional application No. 60/940,475, filed on May 29, 2007.

(51) Int. Cl. A61K 31/4164; C07D 233/54; A61P 1/00

(52) U.S. Cl. 514/397; 548/315.1

(57) ABSTRACT

The present invention relates to novel imidazole compounds having a positive allosteric GABA_{B} receptor (CUR) modulator effect, methods for the preparation of said compounds and to their use, optionally in combination with a GABA_{B} agonist, for the inhibition of transient lower esophageal sphincter relaxations, for the treatment of gastroesophageal reflux disease, as well as for the treatment of functional gastrointestinal disorders and irritable bowel syndrome (IBS).

### FIELD OF THE INVENTION

The present invention relates to novel imidazole compounds having a positive allosteric GABA_{B} receptor (GBR) modulator effect, methods for the preparation of said compounds and their use for the inhibition of transient lower esophageal sphincter relaxations, for the treatment of gastroesophageal reflux disease, as well as for the treatment of functional gastrointestinal disorders and irritable bowel syndrome (IBS).

### BACKGROUND OF THE INVENTION

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastroesophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, recent research (e.g. Holloway & Dent (1990) Gastroenterol. Clin. N. Amer. 19, pp. 517-535) has shown that most reflux episodes occur during transient lower esophageal sphincter relaxations (TLESR), i.e. relaxations not triggered by swallows. It has also been shown that gastric acid secretion usually is normal in patients with GERD.

Consequently, there is a need for a therapy that reduces the incidence of TLESR and thereby prevents reflux.

GABA_{B}-receptor agonists have been shown to inhibit TLESR, which is disclosed in WO 98/11885 A1.

Functional gastrointestinal disorders, such as functional dyspepsia, can be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders: Diagnosis, Pathophysiology and Treatment. 2 ed. McLean, V A: Degnon Associates, Inc.; 2000:351-432 and Drossman D A, Corazziari E, Talley N J, Thompson W G and Whitehead W E. Rome II: A multinational consensus document on Functional Gastrointestinal Disorders. Gut 45(Suppl. 2), III-II81.9-1-1999.

Irritable bowel syndrome (IBS) can be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders: Diagnosis, Pathophysiology and Treatment. 2 ed. McLean, V A: Degnon Associates, Inc.; 2000:351-432 and Drossman D A, Corazziari E, Talley N J, Thompson W G and Whitehead W E. Rome II: A multinational consensus document on Functional Gastrointestinal Disorders. Gut 45(Suppl. 2), III-II81.9-1-1999.

GABA_{B} Receptor Agonists

GABA (4-aminobutanoic acid) is an endogenous neurotransmitter in the central and peripheral nervous systems. Receptors for GABA have traditionally been divided into GABA_{A} and GABA_{B} receptor subtypes. GABA_{B} receptors belong to the superfamily of G-protein coupled receptors (GPCRs).

The most studied GABA_{B} receptor agonist baclofen (4-amino-3-(p-chlorophenyl)butanoic acid; disclosed in CH 449046) is useful as an antispastic agent.

WO 2006/001750 discloses imidazole variants as modulators of GABA receptor for the treatment of GI disorders.

Positive Allosteric Modulation of GABA_{B} Receptors

2,6-Di-tert-butyl-4-(3-hydroxy-2,2-dimethylpropyl)phenol (CGP7930) ad 3-(3,5-di-tert-butyl-4-hydroxyphenyl)-2,2-dimethylpropanal (disclosed in U.S. Pat. No. 5,304,685) have been described to exert positive allosteric modulation of native and recombinant GABA_{B} receptor activity (Society for Neuroscience, 30^{th} Annual Meeting, New Orleans, La., Nov. 4-9, 2000: Positive Allosteric Modulation of Native and Recombinant GABA_{B} Receptor Activity, S. Urwyler et al.; Molecular Pharmacol. (2001), 60, 963-971).

N,N-Dicyclopentyl-2-methylsulfanyl-5-nitro-pyrimidine-4,6-diamine has been described to exert positive allosteric modulation of the GABAB receptor (The Journal of Pharmacology and Experimental Therapeutics, 307 (2003), 322-330).

1H-imidazole-5-carboxylic acid Derivatives

A few 4-amino-1H-imidazole-5-carboxylic acid ethyl esters are disclosed as intermediates for the synthesis of purines (Tetrahedron Lett. (1966), 1885-1889) or imidazo[4,5-d]pyrimidones and imidazo[4,5,-b]pyridines (Monatshefte für Chemie (1976), 107:1413-1421). Also, 1,7-dihydro-6H-purine-6-ones are prepared from 4-acylamino-1H-imidazole-5-carboxylic acid ethyl esters (Tetrahedron (1982), 38:1435-1441). However, these compounds are not known as positive allosteric modulators of the GABA_{B} receptor and have not been described as being useful for the treatment of GERD or functional gastrointestinal disorders.

For a recent review on allosteric modulation of GPCRs, see: Expert Opin. Ther. Patents (2001), 11, 1889-1904.

### OUTLINE OF THE INVENTION

The present invention provides one or more of the following compounds:

Tert-butyl 4-({[3-chloro-4-(isopropylsulfonyl)-2-thienyl]carbonyl}amino)-1-(4-fluorophenyl)-2-methoxy-1H-imidazole-5-carboxylate;

Tert-butyl 4-[(4-chlorobenzoyl)amino]-1-(4-fluorophenyl)-2-methoxy-1H-imidazole-5-carboxylate;

Tert-butyl 4-[(2,3-dihydro-1,4-benzodioxin-2-ylcarbonyl)amino-]1-(4-fluorophenyl)-2-methoxy-1H-imidazole-5-carboxylate;

Tert-butyl 4-({[1-(4-chlorophenyl)cyclopentyl]carbonyl}amino)-1-(4-fluorophenyl)-2-methoxy-1H-imidazole-5-carboxylate;

Tert-butyl 1-(4-fluorophenyl)-2-methoxy-4-[(3,3,3-trifluoro-2-methoxy-2-phenylpropanoyl)amino]-1H-imidazole-5-carboxylate;

Tert-butyl 4-({[1-(4-chlorophenyl)-5-(trifluoromethyl)-1H-pyrazol-4-yl]carbonyl}amino)-1-(4-fluorophenyl)-2-methoxy-1H-imidazole-5-carboxylate;

Tert-butyl 1-(4-fluorophenyl)-2-methoxy-4-{[(6-phenoxypyridin-3-yl)carbonyl]amino}-1H-imidazole-5-carboxylate;

Tert-butyl 1-(4-fluorophenyl)-2-methoxy-4-[(2,4,6-trifluorobenzoyl)amino]-1H-imidazole-5-carboxylate;

Tert-butyl 4-[(2,4-dichlorobenzoyl)amino]-1-(4-fluorophenyl)-2-methoxy-1H-imidazole-5-carboxylate;

2-(2-methoxyethoxy)-1,1-dimethylethyl 4-[(2,4-dichlorobenzoyl)amino]-1-(4-fluorophenyl)-2-methoxy-1H-imidazole-5-carboxylate;

1,1-dimethyl-2-(2,2,2-trifluoroethoxy)ethyl 4-[(2,4-dichlorobenzoyl)amino]-1-(4-fluorophenyl)-2-methoxy-1H-imidazole-5-carboxylate;

1,1-dimethyl-2-phenoxyethyl 4-[(2,4-dichlorobenzoyl)amino]-1-(4-fluorophenyl)-2-methoxy-1H-imidazole-5-carboxylate;

1-(ethoxycarbonyl)cyclopropyl 4-[(2,4-dichlorobenzoyl)amino]-1-(4-fluorophenyl)-2-methoxy-1H-imidazole-5-carboxylate;

1-(5-ethyl-5-methyltetrahydrofuran-2-yl)-1-methylethyl 4-[(2,4-dichlorobenzoyl)amino]-1-(4-fluorophenyl)-2-methoxy-1H-imidazole-5-carboxylate;

2-methoxy-1,1-dimethylethyl 1-(1,3-benzodioxol-5-yl)-4-[(2,3-dihydro-1,4-benzodioxin-2-ylcarbonyl)amino]-2-methoxy-1H-imidazole-5-carboxylate;

2-methoxy-1,1-dimethylethyl 1-(1,3-benzodioxol-5-yl)-4-[(4-chlorobenzoyl)amino]-2-methoxy-1H-imidazole-5-carboxylate;

2-methoxy-1,1-dimethylethyl 1-(1,3-benzodioxol-5-yl)-4-{[(benzyloxy)acetyl]amino}-2-methoxy-1H-imidazole-5-carboxylate;

2-methoxy-1,1-dimethylethyl 4-[(2,4-dichlorobenzoyl)amino]-2-methoxy-1-(3-methoxyphenyl)-1H-imidazole-5-carboxylate;

Tert-butyl 4-[(4-azidobenzoyl)amino]-2-ethyl-1-(4-fluorophenyl)-1H-imidazole-5-carboxylate; and

Tert-butyl 4-[(2,4-dichlorobenzoyl)amino]-2-ethyl-1-(4-fluorophenyl)-1H-imidazole-5-carboxylate;

or a pharmaceutically acceptable salt thereof. These compounds will hereinafter be referred to as the compounds of the invention.

The compounds of the invention are useful as positive allosteric GABA_{B} receptor modulators.

When the compounds of the invention have at least one asymmetric carbon atom, they can exist in several stereochemical forms. The present invention includes the mixture of isomers as well as the individual stereoisomers. The present invention further includes geometrical isomers, rotational isomers, enantiomers, racemates and diastereomers.

Where applicable, the compounds of the invention may be used in neutral form, e.g. as a carboxylic acid, or in the form of a salt, preferably a pharmaceutically acceptable salt such as the sodium, potassium, ammonium, calcium or magnesium salt of the compound at issue.

The compounds of the invention are useful as positive allosteric GBR (GABA_{B} receptor) modulators. A positive allosteric modulator of the GABA_{B} receptor is defined as a compound which makes the GABA_{B} receptor more sensitive to GABA and GABA_{B} receptor agonists by binding to the GABA_{B} receptor protein at a site different from that used by the endogenous ligand. The positive allosteric GBR modulator acts synergistically with an agonist and increases potency an&/or intrinsic efficacy of the GABA_{B} receptor agonist It has also been shown that positive allosteric modulators acting at the GABA_{B} receptor can produce an agonistic effect. Therefore, compounds of the invention can be effective as full or partial agonists.

A further aspect is a compound of the invention for use in therapy.

As a consequence of the GABA_{B} receptor becoming more sensitive to GABA_{B} receptor agonists upon the administration of a positive allosteric modulator, an increased inhibition of transient lower esophageal sphincter relaxations (TLESR) for a GABA_{B} agonist is observed. Consequently, the present invention is directed to the use of a positive allosteric GABA_{B} receptor modulator according to compounds of the invention, optionally in combination with a GABA_{B} receptor agonist, for the preparation of a medicament for the inhibition of transient lower esophageal sphincter relaxations (TLESRs).

A further aspect is the use of a compound of the invention, optionally in combination with a GABA_{B} receptor agonist, for the manufacture of a medicament for the prevention of reflux. Another aspect of the invention is a compound of the invention, optionally in combination with a GABA_{B} receptor agonist, for use in the prevention of reflux.

Still a further aspect is the use of a compound of the invention, optionally in combination with a GABA_{B} receptor agonist, for the manufacture of a medicament for the treatment of gastroesophageal reflux disease (GERD). Another aspect of the invention is a compound of the invention, optionally in combination with a GABA_{B} receptor agonist, for use in the treatment of gastroesophageal reflux disease (GERD).

Effective management of regurgitation in infants would be an important way of preventing, as well as curing lung disease due to aspiration of regurgitated gastric contents, and for managing failure to thrive, inter alia due to excessive loss of ingested nutrient. Thus, a further aspect is the use of a compound of the invention, optionally in combination with a GABA_{B} receptor agonist, for the manufacture of a medicament for the treatment of lung disease. Another aspect of the invention is a compound of the invention, optionally in combination with a GABA_{B} receptor agonist, for use in the treatment of lung disease.

Still another aspect is the use of a compound of the invention, optionally in combination with a GABA_{B} receptor agonist, for the manufacture of a medicament for the management of failure to thrive. Another aspect of the invention is a compound of the invention, optionally in combination with a GABA_{B} receptor agonist, for use in the management of failure to thrive.

Still another aspect is the use of a compound of the invention, optionally in combination with a GABA_{B} receptor agonist, for the manufacture of a medicament for the treatment or prevention of asthma, such as reflux-related asthma. Another aspect of the invention is a compound of the invention, optionally in combination with a GABA_{B} receptor agonist, for use in the treatment or prevention of asthma, such as reflux-related asthma.

A further aspect is the use of a compound of the invention, optionally in combination with a GABA_{B} receptor agonist, for the manufacture of a medicament for the treatment or prevention of laryngitis or chronic laryngitis. Another aspect of the invention is a compound of the invention, optionally in combination with a GABA_{B} receptor agonist, for use in the treatment or prevention of laryngitis or chronic laryngitis.

A further aspect of the present invention is a method for the inhibition of transient lower esophageal sphincter relaxations (TLESRs), whereby a pharmaceutically and pharmacologically effective amount of a compound of the invention, optionally in combination with a GABA_{B} receptor agonist, is administered to subject in need of such inhibition.

Another aspect is a method for the prevention of reflux, whereby a pharmaceutically and pharmacologically effective amount of a compound of the invention, optionally in combination with a GABA_{B} receptor agonist, is administered to a subject in need of such prevention.

Still a further aspect is a method for the treatment of gastroesophageal reflux disease (GERD), whereby a pharmaceutically and pharmacologically effective amount of a compound of the invention, optionally in combination with a GABA_{B} receptor agonist, is administered to a subject in need of such treatment.

Another aspect is a method for the treatment or prevention of regurgitation, whereby a pharmaceutically and pharmacologically effective amount of a compound of the invention, optionally in combination with a GABA_{B} receptor agonist, is administered to a subject in need of such treatment.

Yet another aspect is a method for the treatment or prevention of regurgitation in infants, whereby a pharmaceutically and pharmacologically effective amount of a compound of the invention, optionally in combination with a GABA_{B} receptor agonist, is administered to a subject in need of such treatment.

Still a further aspect is a method for the treatment, prevention or inhibition of lung disease, whereby a pharmaceutically and pharmacologically effective amount of a compound of the invention, optionally in combination with a GABA_{B} receptor agonist, is administered to a subject in need of such treatment. The lung disease to be treated may inter alia be due to aspiration of regurgitated gastric contents.

Still a further aspect is a method for the management of failure to thrive, whereby a pharmaceutically and pharmacologically effective amount of a compound of the invention, optionally in combination with a GABA_{B} receptor agonist, is administered to a subject in need of such treatment.

A further aspect is a method for the treatment or prevention of asthma, such as reflux-related asthma, whereby a pharmaceutically and pharmacologically effective amount of a compound of the invention, optionally in combination with a GABA_{B} receptor agonist, is administered to a subject in need of such treatment.

A further aspect is a method for the treatment or prevention of laryngitis or chronic laryngitis, whereby a pharmaceutically and pharmacologically effective amount of a compound of the invention, optionally in combination with a GABA_{B} receptor agonist, is administered to a subject in need of such treatment.

A further embodiment is the use of a compound of the invention, optionally in combination with a GABA_{B} receptor agonist, for the manufacture of a medicament for the treatment of a functional gastrointestinal disorder (FGD). Another aspect of the invention is a compound of the invention, optionally in combination with a GABA_{B} receptor agonist, for use in the treatment of a functional gastrointestinal disorder (FGD). Still another aspect is a method for the treatment of a functional gastrointestinal disorder, whereby an effective amount of a compound of the invention, optionally in combination with a GABA_{B} receptor agonist, is administered to a subject suffering from said condition.

A further embodiment is the use of a compound of the invention, optionally in combination with a GABA_{B} receptor agonist, for the manufacture of a medicament for the treatment of functional dyspepsia. Another aspect of the invention is a compound of the invention, optionally in combination with a GABA_{B} receptor agonist, for use in the treatment of functional dyspepsia. Still another aspect is a method for the treatment of functional dyspepsia, whereby an effective amount of a compound of the invention, optionally in combination with a GABA_{B} receptor agonist, is administered to a subject suffering from said condition.

Functional dyspepsia refers to pain or discomfort centered in the upper abdomen. Discomfort may be characterized by or combined with upper abdominal fullness, early satiety, bloating or nausea. Etiologically, patients with functional dyspepsia can be divided into two groups:
1—Those with an identifiable pathophysiological or microbiologic abnormality of uncertain clinical relevance (e.g. Helicobacter pylori gastritis, histological duodenitis, gallstones, visceral hypersensitivity, gastroduodenal dysmotility)
2—Patients with no identifiable explanation for the symptoms.

Functional dyspepsia can be diagnosed according to the following. At least 12 weeks, which need not be consecutive within the preceding 12 months of
1—Persistent or recurrent dyspepsia (pain or discomfort centered in the upper abdomen) and
2—No evidence of organic disease (including at upper endoscopy) that is likely to explain the symptoms and
3—No evidence that dyspepsia is exclusively relieved by defecation or associated with the onset of a change in stool frequency or form.

Functional dyspepsia can be divided into subsets based on distinctive symptom patterns, such as ulcer-like dyspepsia, dysmotility-like dyspepsia and unspecified (non-specific) dyspepsia.

Currently existing therapy of functional dyspepsia is largely empirical and directed towards relief of prominent symptoms. The most commonly used therapies still include antidepressants.

A further aspect is the use of a compound according to the invention, optionally in combination with a GABA_{B} receptor agonist, for the manufacture of a medicament for the treatment or prevention of irritable bowel syndrome (IBS), such as constipation predominant IBS, diarrhea predominant IBS or alternating bowel movement predominant IBS Another aspect of the invention is a compound of the invention, optionally in combination with a GABA_{B} receptor agonist, for use in the treatment or prevention of irritable bowel syndrome (IBS), such as constipation predominant IBS, diarrhea predominant IBS or alternating bowel movement predominant IBS.

A further aspect is a method for the treatment or prevention of irritable bowel syndrome (IBS), whereby a pharmaceutically and pharmacologically effective amount of a compound of the invention, optionally in combination with a GABA_{B} receptor agonist, is administered to a subject in need of such treatment.

IBS is herein defined as a chronic functional disorder with specific symptoms that include continuous or recurrent abdominal pain and discomfort accompanied by altered bowel function, often with abdominal bloating and abdominal distension. It is generally divided into 3 subgroups according to the predominant bowel pattern:
1—diarrhea predominant
2—constipation predominant
3—alternating bowel movements.

Abdominal pain or discomfort is the hallmark of IBS and is present in the three subgroups. IBS symptoms have been categorized according to the Rome criteria and subsequently modified to the Rome II criteria. This conformity in describing the symptoms of IBS has helped to achieve consensus in designing and evaluating IBS clinical studies. The Rome II diagnostic criteria are:
1—Presence of abdominal pain or discomfort for at least 12 weeks (not necessarily consecutively) out of the preceding year
2—Two or more of the following symptoms:
a) Relief with defecation
b) Onset associated with change in stool frequency
c) Onset associated with change in stool consistency

A further aspect is the use of a compound according to the invention, optionally in combination with a GABA_{B} receptor agonist, for the manufacture of a medicament for the treatment or prevention of CNS disorders, such as anxiety. Another aspect of the invention is a compound of the invention, optionally in combination with a GABA_{B} receptor agonist, for use in the treatment or prevention of CNS disorders, such as anxiety.

A further aspect is a method for the treatment or prevention of CNS disorders, such as anxiety whereby a pharmaceutically and pharmacologically effective amount of a compound of the invention, optionally in combination with a GABA_{B} receptor agonist, is administered to a subject in need of such treatment.

A further aspect is the use of a compound of the invention, optionally in combination with a GABA_{B} receptor agonist, for the manufacture of a medicament for the treatment or prevention of depression. Another aspect of the invention is a compound of the invention, optionally in combination with a GABA_{B} receptor agonist, for use in the treatment or prevention of depression.

A further aspect is a method for the treatment or prevention of depression, whereby a pharmaceutically and pharmacologically effective amount of a compound of the invention, optionally in combination with a GABA_{B} receptor agonist, is administered to a subject in need of such treatment.

For the purpose of this invention, the term “agonist” should be understood as including full agonists as well as partial agonists, whereby a “partial agonist” should be understood as a compound capable of partially, but not fully, activating GABA_{B} receptors.

The wording “TLESR”, transient lower esophageal sphincter relaxations, is herein defined in accordance with Mittal, R. K., Holloway, R. H., Penagini, R., Blackshaw, L. A., Dent, J., 1995; Transient lower esophageal sphincter relaxation. Gastroenterology 109, pp. 601-610.

The wording “reflux” is defined as a condition when fluid from the stomach is being able to pass into the esophagus, since the mechanical barrier (the esophageal sphincter) is temporarily not functioning as desired at such times.

The wording “GERD”, gastroesophageal reflux disease, is defined in accordance with van Heerwarden, M. A., Smout A. J. P. M., 2000; Diagnosis of reflux disease. Bailière's Clin. Gastroenterol. 14, pp. 759-774.

A “combination” according to the invention may be present as a “fix combination” or as a “kit of parts combination”.

A “fix combination” is defined as a combination wherein (i) a compound of the invention; and (ii) a GABA_{B} receptor agonist are present in one unit. One example of a “fix combination” is a pharmaceutical composition wherein (i) a compound of the invention and (ii) a GABA_{B} receptor agonist are present in admixture. Another example of a “fix combination” is a pharmaceutical composition wherein (i) a compound of the invention and (ii) a GABA_{B} receptor agonist; are present in one unit without being in admixture.

A “kit of parts combination” is defined as a combination wherein (i) a compound of the invention and (ii) a GABA_{B} receptor agonist are present in more than one unit. One example of a “kit of parts combination” is a combination wherein (i) a compound of the invention and (ii) a GABA_{B} receptor agonist are present separately. The components of the “kit of parts combination” may be administered simultaneously, sequentially or separately, i.e. separately or together.

The term “positive allosteric modulator” is defined as a compound which makes a receptor more sensitive to receptor agonists by binding to the receptor protein at a site different from that used by the endogenous ligand.

The term “therapy” and the term “treatment” also include “prophylaxis” and/or prevention unless stated otherwise. The terms “therapeutic” and “therapeutically” should be construed accordingly.

Pharmaceutical Formulations

The compounds of the invention can be formulated alone or in combination with a GABA_{B} receptor agonist.

For clinical use, the compounds of the invention, optionally in combination with a GABA_{B} receptor agonist, is in accordance with the present invention suitably formulated into pharmaceutical formulations for oral administration. Also rectal, parenteral or any other route of administration may be contemplated to the skilled man in the art of formulations. Thus, the compound of the invention, optionally in combination with a GABA_{B} receptor agonist, is formulated with a pharmaceutically and pharmacologically acceptable carrier or adjuvant. The carrier may be in the form of a solid, semi-solid or liquid diluent.

In the preparation of oral pharmaceutical formulations in accordance with the invention, the compound of the invention, optionally in combination with a GABA_{B} receptor agonist, to be formulated is mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or compressed into tablets.

Soft gelatine capsules may be prepared with capsules containing a mixture of a compound of the invention, optionally in combination with a GABA_{B} receptor agonist, with vegetable oil, fat, or other suitable vehicle for soft gelatine capsules. Hard gelatine capsules may contain a compound of the invention, optionally in combination with a GABA_{B} receptor agonist, in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance(s) mixed with a neutral fat base; (ii) in the form of a gelatine rectal capsule which contains a compound of the invention, optionally in combination with a GABA_{B} receptor agonist, in a mixture with a vegetable oil, paraffin oil, or other suitable vehicle for gelatine rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing a compound of the invention, optionally in combination with a GABA_{B} receptor agonist, and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agents. Liquid preparations for oral administration may also be prepared in the form of a dry powder to be reconstituted with a suitable solvent prior to use.

Solutions for parenteral administration may be prepared as a solution of a compound of the invention, optionally in combination with a GABA_{B} receptor agonist, in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

In one aspect of the present invention, a compound of the invention, optionally in combination with a GABA_{B} receptor agonist, may be administered once or twice daily, depending on the severity of the patient's condition. A typical daily dose of the compounds of the invention is from 0.1 to 100 mg per kg body weight of the subject to be treated, but this will depend on various factors such as the route of administration, the age and weight of the patient as well as of the severity of the patient's condition.

Methods of Preparation

The compounds of the invention of the present invention may be prepared as illustrated below. The compounds may also be prepared as described for structurally related compounds in the prior art. The reactions can be carried out according to standard procedures or as described in the experimental section.

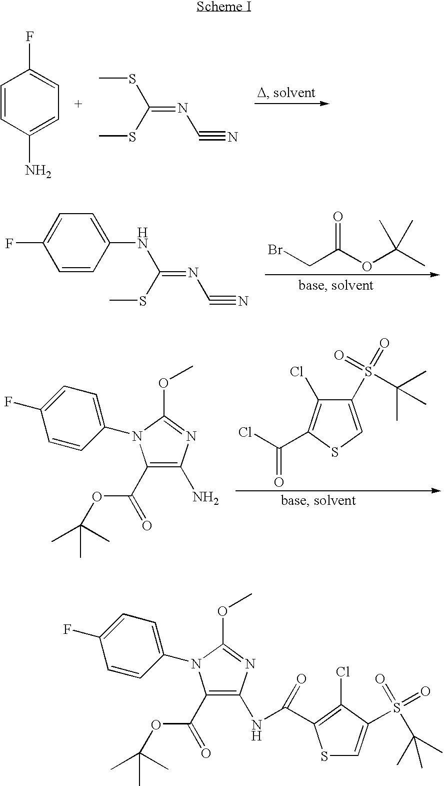

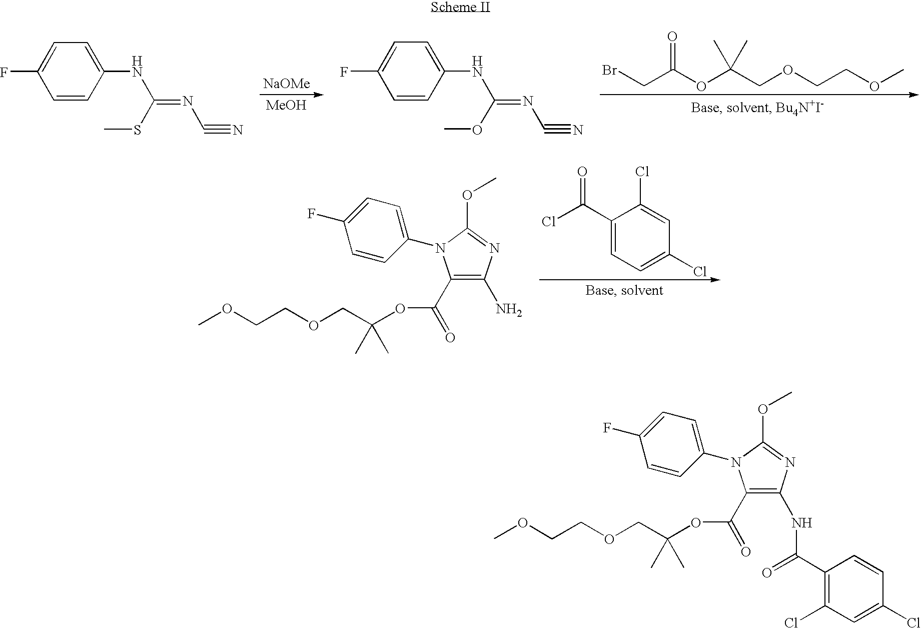

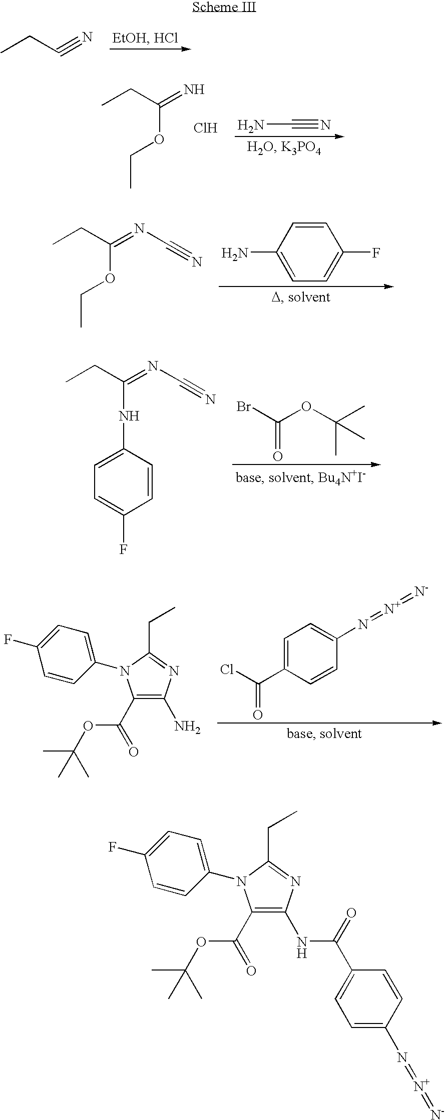

It shall be understood that the individual reaction steps in the schemes above may require a reaction temperature deviating from room temperature. Heating may be achieved using conventional methods such as heating the reaction mixture on a oil bath or heating the reaction mixture in a microwave oven. Cooling may be achieved using conventional methods such as cooling the reaction mixture on an ice bath, cooling with solid carbon dioxide in an appropriate solvent or by using a cryostatic temperature regulator.

In the schemes above the expression “solvent” refers to a solvent which does not react with the starting materials, reagents, intermediates or products in a manner which adversely affects the yield of the desired product. Examples of such solvents are for instance dimethylformamide, methylene chloride and acetonitrile.

The compounds of the invention may be isolated from their reaction mixtures using conventional techniques.

Persons skilled in the art will appreciate that, in order to obtain compounds of the invention in an alternative and in some occasions more convenient manner, the individual process steps mentioned hereinbefore may be performed in a different order, and/or the individual reactions may be performed at a different stage in the overall route.

### EXAMPLES

Abbreviations

DCM dichloromethane

DMF N,N′-dimethylformamide

DMSO Dimethyl sulphoxide

DIBAL-H diisobutylaluminium hydride

DIPEA N,N′-diisopropylethylamine

EtOAc ethyl acetate

HPFC high performance flash chromatography

HPLC high performance liquid chromatography

HRMS high resolution mass spectroscopy

LC-MS liquid chromatography mass spectroscopy

MeCN acetonitrile

NMR nuclear magnetic resonance

Tert tertiary

TBME methyl tert-butyl ether

THF tetrahydrofuran

h hour(s)

min minutes

br broad

s singlet

d doublet

t triplet

q quartet

m multiplet

dd double doublet

td triple doublet

General Experimental Procedures

Phase Separator from IST was used. Flash column chromatography employed normal phase silica gel 60 (0.040-0.063 mm, Merck) or IST Isolute®SPE columns normal phase silica gel or Biotage Horizon™ HPFC System using silica FLASH+™ HPFC™ Cartridges. HPLC purifications were performed on either a Gilson preparative HPLC system with gradient pump system 333/334, GX-281 injector, UV/VIS detector 155. Trilution LC v.1.4 software. In acidic system equipped with an Kromasil C8 10 μm 250×20 ID mm column or Kromasil C8 10 μm 250×50 ID mm column and as gradient: mobile phase (buffer): H₂O/MeCN/FA 95/5/0.2 and mobile phase (organic): MeCN. In neutral system equipped with an Kromasil C8 10 μm 250×20 ID mm column or Kromasil C8 10 μm 250×50 ID mm column and as gradient: mobile phase (buffer): MeCN/0,1M NH₄OAc 5/95 and mobile phase (organic): MeCN. In basic system system equipped with an XBridge C18 10 μm 250×19 ID mm column or XBridge C18 10 μm 250×50 ID mm column and as gradient: mobile phase (buffer): H₂O/MeCN/NH₃ 95/5/0.2 and mobile phase (organic): MeCN. Or on a Waters preparative HPLC system equipped with a Kromasil C8 10 mm 250 mm×21.2 mm column and a gradient mobile phase (buffer): MeCN/0.1M NH₄OAc 5/95 and mobile phase (organic): MeCN or on a Waters FractionLynx HPLC system with a mass triggered fraction collector, equipped with a Xbridge Prep C18 5μ 19 mm×150 mm column using MeCN/NH3 buffer system with a gradient from 95% mobilphase A (0.2% NH3 in water, pH10) to 95% mobilphase B (100% MeCN) unless otherwise stated. ¹H NMR and ¹³C NMR measurements were performed on a BRUKER ACP 300 or on a Varian Inova 400, 500 or 600 spectrometer, operating at ¹H frequencies of 300, 400, 500, 600 MHz, respectively, and ¹³C frequencies of 75, 100, 125 and 150 MHz, respectively. Chemical shifts are given in δ values (ppm) with the solvents used as internal standard, unless otherwise stated. Microwave heating was performed using single node heating in a Smith Creator or Emrys Optimizer from Personal Chemistry, Uppsala, Sweden. Mass spectral data were obtained using a Micromass LCT or Waters Q-Tof micro system and, where appropriate, either positive ion data or negative ion data were collected.

Compound names were generated by ACD/Name Release 9.0. Product Version: 9.04 (Build 6210, 20 Jul. 2005)

The GTPγS values (IC₅₀ in μM) mentioned in the examples below were measured by the method described later starting on page 42.

### Example 1

### Tert-butyl 4-({[3-chloro-4-(isopropylsulfonyl)-2-thienyl]carbonyl}amino)-1-(4-fluorophenyl)-2-methoxy-1H-imidazole-5-carboxylate

### Step a: Methyl N′-cyano-N-(4-fluorophenyl)imidothiocarbamate

A mixture of 4-fluoroaniline (4.0 g 36.0 mmol) and dimethyl N-cyanoiminodithiocarbonate (5.26 g, 36.0 mmol) in ethanol (100 ml) was heated to reflux for 16 h. The product was collected by filtration and washed with heptane (4.70 g, 62%).

¹H NMR (400 MHz, DMSO) δ 10.20-10.00 (br, 1H), 7.47-7.39 (m, 2H), 7.25-7.16 (m, 2H), 2.65 (s, 3H).

### Step b: Tert-butyl 4amino-1-(4-fluorophenyl)-2-methoxy-1H-imidazole-5-carboxylate

Tert-butyl bromoacetate (6.57 g, 33.69 mmol) was added dropwise to a mixture of methyl N′-cyano-N-(4-fluorophenyl)imidothiocarbamate (4.70 g, 22.46 mmol) and potassium carbonate (4.67 g, 33.76 mmol) in DMF (40 ml). The mixture was heated to 80° C. for 2 h and then cooled to 0° C. Sodium methoxide (45 ml, 0.5 M in methanol) was added. The reaction was continued at 0° C. for 20 min and then more sodium methoxide (45 ml, 0.5 M in methanol) was added. The reaction was continued at 0° C. for 10 min and then at room temperature for 1 h. DCM and water was added. Most of the solvent was evaporated. DCM was added, the phases separated the organic phase washed with water and dried over magnesium sulfate. The product was purified further by preparatory HPLC (kromasil C8 column, ammonium acetate (aq, 0.1 M):MeCN, product came at 86% MeCN) (2.66 g, 39%).

¹H NMR (400 MHz, CDCl₃) δ 7.22-7.14 (m, 2H), 7.11-7.02 (m, 2H), 5.10-4.90 (br, 2H), 3.93 (s, 3H), 1.25 (s, 9H).

MS m/z 308 (M+H)⁺.

### Step c: Tert-butyl 4-({[3-chloro-4-(isopropylsulfonyl)-2-thienyl]carbonyl}amino)-1-(4-fluorophenyl)-2-methoxy-1H-imidazole-5-carboxylate

A mixture of 3-chloro-4-(isopropylsulphonyl)thiophene-2-carbonyl chloride (489 mg, 1.70 mmol) in DCM (20 ml) was added to a mixture of tert-butyl 4-amino-1-(4-fluorophenyl)-2-methoxy-1H-imidazole-5-carboxylate (420 mg, 1.37 mmol) and potassium carbonate (2.0 g, 14.5 mmol) in DCM/water (1:1, 40 ml). The reaction was continued at room temperature for 3 h. The phases were separated and the product purified further by preparatory HPLC (kromasil C8 column, ammonium acetate (aq, 0.1 M):MeCN, product came at 97% MeCN) to give a powder after freeze drying (632 mg, 83%).

¹H NMR (400 MHz, CDCl₃) δ 10.59 (s, 1H), 8.34 (s, 1H), 7.26-7.08 (m, 4H), 4.07 (s, 3H), 3.60-3.48 (m, 1H), 1.35 (d, 6H), 1.17 (s, 9H).

HRMS Calcd for [C₂₃H₂₅ClFN₃O₆S₂+H]+: 558.094. Found: 558.095.

GTPγS(IC₅₀): 4 μM

### Example 2

### Tert-butyl 4-[(4-chlorobenzoyl)amino]-1-(4-fluorophenyl)-2-methoxy-1H-imidazole-5-carboxylate

A mixture of 4-chlorobenzoyl chloride (63 m,0.18 mmol), tert-butyl 4-amino-1-(4-fluorophenyl)-2-methoxy-1H-imidazole-5-carboxylate (prepared as described in Example 1 step a-b, 46 mg, 0.15 mmol) and polymer supported diisopropylethylamine (3.88 mmol/g, 77 mg) in THF (2 ml) was stirred at room temperature for 17 h. The mixture was filtered, evaporated and purified by preparatory HPLC (Sunfire C18 column, ammonium acetate (aq, 0.1 M):MeCN) (0.7 mg, 1.1%).

¹H NMR (400 MHz, CDCl₃) δ 10.25-10.15 (br, 1H), 7.91 (d, 2H), 7.45 (d, 2H), 7.25-7.07 (m, 4H), 4.10 (s, 3H), 1.20 (s, 9H).

MS m/z 446, 448 (M+H)⁺

GTPγS(IC₅₀): 3.1 μM

Examples 3-8 were prepared in an analogous method to Example 2.

### Example 3

### Tert-butyl 4-[(2,3-dihydro-1,4-benzodioxin-2-ylcarbonyl)amino]-1-(4-fluorophenyl)-2-methoxy-1H-imidazole-5-carboxylate

(22 mg, 31%)

¹H NMR (400 MHz, CDCl₃) δ 10.25-10.15 (br, 1H), 7.11-6.94 (m, 5H), 6.81-6.75 (m, 3H), 4.75-4.65 (m, 1H), 4.52-4.47 (m, 1H), 4.25-4.19 (m, 1H), 3.95 (s, 3H), 1.12 (s, 9H).

MS m/z 470 (M+H)⁺

GTPγS(IC₅₀): 1.5 μM

### Example 4

### Tert-butyl 4-({[1-(4-chlorophenyl)cyclopentyl]carbonyl}amino)-1-(4-fluorophenyl)-2-methoxy-1H-imidazole-5-carboxylate

(1.3 mg, 1.7%)

¹H NMR (400 MHz, CDCl₃) δ 9.45-9.40 (br, 1H), 7.44-7.26 (m, 4H), 7.13-7.02 (m, 4H), 4.03 (s, 3H), 2.70-1.20 (m, 8H), 1.11 (s, 9H).

MS m/z 514, 516 (M+H)⁺

GTPγS(IC₅₀): 1.4 μM

### Example 5

### Tert-butyl 1-(4-fluorophenyl)-2-methoxy4-[(3,3,3-trifluoro-2-methoxy-2-phenylpropanoyl)amino]-1H-imidazole-5-carboxylate

(46 mg, 58%)

¹H NMR (400 MHz, CDCl₃) δ 10.26-10.19 (br, 1H), 7.72-7.64 (m, 2H), 7.42-7.35 (m, 3H), 7.19-7.05 (m, 4H), 4.05 (s, 3H), 3.58 (s, 3H), 1.22 (s, 9H).

MS m/z 524 (M+H)⁺

GTPγS(IC₅₀): 6.2 μM

### Example 6

### Tert-butyl 4-({[1-(4-chlorophenyl)-5-(trifluoromethyl)-1H-pyrazol-4-yl]carbonyl}amino)-1-(4-fluorophenyl)-2-methoxy-1H-imidazole-5-carboxylate

(13 mg, 15%)

¹H NMR (400 MHz, CDCl₃) δ 9.90-9.80 (br, 1H), 8.03 (s, 1H), 7.50-7.37 (m, 4H), 7.23-7.07 (m, 4H), 4.06 (s, 3H), 1.19 (s, 9H).

MS m/z 580, 582 (M+H)⁺

GTPγS(IC₅₀): 1.6 μM

### Example 7

### Tert-butyl 1-(4-fluorophenyl)-2-methoxy-4-{[(6-phenoxypyridin-3-yl)carbonyl]amino}-1H-imidazole-5-carboxylate

(2.7 mg, 3.5%)

¹H NMR (400 MHz, CDCl₃) δ 10.29-10.24 (br, 1H), 8.81 (d, 1H), 8.30 (dd, 1H), 7.45-7.39 (m, 2H), 7.27-7.07 (m, 7H), 7.00 (d, 1H), 4.09 (s, 3H), 1.18 (s, 9H).

MS m/z 505 (M+H)⁺

GTPγS(IC₅₀): 1.6 μM

### Example 8

### Tert-butyl 1-(4-fluorophenyl)-2-methoxy-4-[(2,4,6-trifluorobenzoyl)amino]-1H-imidazole-5-carboxylate

(35 mg, 20%)

¹H-NMR (400 MHz, CDCl₃) δ 9.67 (br, 1H), 7.30-6.93 (m, 4H), 6.80-6.54 (m, 2H), 4.04 (br, 3H), 1.16 (br, 9H).

HRMS Calcd for [C₂₂H₁₉F₄N₃O₄+H]⁺: 466.139. Found: 466.138.

GTPγS(IC₅₀): 0.81≈0.8 μM

### Example 9

### Tert-butyl 4-[(2,4-dichlorobenzoyl)amino]-1-(4-fluorophenyl)-2-methoxy-1H-imidazole-5-carboxylate

2,4-Dichlorobenzoyl chloride (0.34 ml, 2.4 mmol) was added dropwise to a solution of tert-butyl 4-amino-1-(4-fluorophenyl)-2-methoxy-1H-imidazole-5-carboxylate (prepared as described in Example 1 step a-b, 615 mg, 2.00 mmol) and DIPEA (0.46 ml, 2.6 mmol) in DCM (20 ml) at 0° C. The reaction mixture was allowed to reach ambient temperature. After stirring at ambient temperature for 7 h, new portions of DIPEA (0.23 ml, 1.3 mmol) and 2,4-dichlorobenzoyl chloride (0.17 ml, 1.2 mmol) were added and the stirring was continued for 64 h. The mixture was washed with water and the organic phase was dried over magnesium sulfate. The residue was purified by column chromatography (silica gel; EtOAc/DCM, 1:0 to 23:2) to yield a solid (534 mg, 56%).

¹H-NMR (500 MHz, CDCl₃): δ 9.83 (br, 1H), 7.70 (br, 1H), 7.48 (s, 1H), 7.36 (d, 1H), 7.26-7.19 (m, 2H), 7.19-7.10 (m, 2H), 4.08 (br, 3H), 1.22 (s, 9H).

HRMS Calcd for [C₂₂H₂₀Cl₂FN₃O₄+H]⁺: 480.089. Found; 480.088.

GTPγS(IC₅₀): 2.16≈2.2 μM

### Example 10

### 2-(2-methoxyethoxy)-1,1-dimethylethyl 4-[(2,4-dichlorobenzoyl)amino]-1-(4-fluorophenyl)-2-methoxy-1H-imidazole-5-carboxylate

### Step a: Methyl N′-cyano-N-(4-fluorophenyl)imidocarbamate

Methyl N′-cyano-N-(4-fluorophenyl)imidothiocarbamate (prepared as described in Example 1 step a, 1.05 g, 5.0 mmol) was suspended in sodium methoxide (20 ml, 0.5 M in methanol). The mixture was refluxed for 6 h. The reaction was quenched with acetic acid. The product was collected by filtration and washed with water (880 mg, 91%).

¹H NMR (400 MHz, CDCl₃) δ 8.36-8.22 (br, 1H), 7.25-7.16 (m, 2H), 7.07-6.98 (s, 2H), 3.88 (s, 3H).

### Step b: 2-(2-methoxyethoxy)-1,1-dimethylethyl bromoacetate

Bromoacetyl chloride (668 μl, 8.10 mmol) was slowly added to 2-methyl-1-phenoxypropan-2-ol (1.0 g, 6.75 mmol) in toluene (10 ml). The mixture was refluxed for 18 h. The solvent was evaporated at reduced pressure (1.8 g crude).

¹H NMR (400 MHz, CDCl₃) δ 3.83 (s, 2H), 3.65-3.49 (m, 4H), 3.36 (s, 5H), 1.43 (s, 6H).

### Step c: 2-(2-methoxyethoxy)-1,1-dimethylethyl 4-amino-1-(4-fluorophenyl)-2-methoxy-1H-imidazole-5-carboxylate

A solution of 2-(2-methoxyethoxy)-1,1-dimethylethyl bromoacetate (1.17 g, crude) in DMF (5 ml) was added dropwise to a mixture of methyl N′-cyano-N-(4-fluorophenyl)imidocarbamate (350 mg, 1.81 mmol), potassium carbonate (300 mg, 2.17 mmol) and tetrabutylammonium iodide (45 mg, 0.122 mmol) in DMF (5 ml). The mixture was reacted at room temperature for 6 h and then cooled to 0° C. Sodium methoxide (3.7 ml, 0.5 M in methanol) was added. The reaction was continued at 0° C. for 5 min and then at room temperature for 40 min. DCM and water was added, the phases were separated and the organic phase washed with water and dried over magnesium sulfate. The product was purified further by preparatory HPLC (kromasil C8 column, ammonium acetate (aq, 0.1 M):MeCN, product came at 90% MeCN) (66 mg, 13% for two steps).

¹H NMR (400 MHz, CDCl₃) δ 7.22-7.14 (m, 2H), 7.08-6.99 (m, 2H), 5.23-5.10 (br, 2H), 3.91 (s, 3H), 3.55-3.45 (m, 4H), 3.39 (s, 2H), 3.32 (s, 3H), 1.32 (s, 6H).

MS m/z 382 (M+H)⁺.

### Step d: 2-(2-methoxyethoxy)-1,1-dimethylethyl 4-[(2,4-dichlorobenzoyl)amino]-1-(4-fluorophenyl)-2-methoxy-1H-imidazole-5carboxylate

Prepared as described in Example 2 (43 mg, 39%).

¹H NMR (400 MHz, CDCl₃) δ 9.78-9.64 (br, 1H), 7.68-7.56 (m, 1H), 7.45-7.40 (m, 1H), 7.34-7.27 (m, 1H), 7.25-7.15 (m, 2H), 7.13-7.05 (m, 2H), 4.14-3.86 (br, 3H), 3.41 (s, 4H), 3.36 (s, 2H), 3.30 (s, 3H), 1.21 (s, 6H).

HRMS Calcd for [C₂₅H₂₆Cl₂ FN₃O₆+H]+: 554.126, Found: 554.130.

GTPγS(IC₅₀): 1.5 μM

Examples 11-14 were prepared in an analogous method to Example 10.

### Example 11

### 1,1-dimethyl-2-(2,2,2-trifluoroethoxy)ethyl 4-[(2,4-dichlorobenzoyl)amino]-1-(4-fluorophenyl)-2-methoxy-1H-imidazole-5-carboxylate

Prepared as described in step c, Example 1 from Tert-butyl 4-amino-1-(4-fluorophenyl)-2-methoxy-1H-imidazole-5-carboxylate of Ex. 14, step b (137 mg, 55%).

¹H NMR (400 MHz, CDCl₃) δ 9.72 (s, 1H), 7.70-7.55 (m, 1H), 7.45-7.42 (m, 1H), 7.35-7.28 (m, 1H), 7.22-7.15 (m, 2H), 7.13-7.05 (m, 2H), 4.01 (s, 3H), 3.75-3.60 (m, 2H), 3.54 (s, 2H) 1.18 (s, 6H).

HRMS Calcd for [C₂₄H₂₁Cl₂F₄N₃O₅+H]+: 578.087+Found: 578.090.

GTPγS(IC₅₀): 1.1 μM

### Example 12

### 1,1-dimethyl-2-phenoxyethyl 4-[(2,4-dichlorobenzoyl)amino]-1-(4-fluorophenyl)-2-methoxy-1H-imidazole-5-carboxylate

### Step a: methyl 2-[(4-methoxybenzyl)oxy]-2-methylpropanoate

2-Hydroxyisobutyric acid methyl ester (1.96 ml, 16.97 mmol) was added slowly to a mixture of sodium hydride (626 mg, 26.08 mmol) and tetrabutylammonium iodide (114 mg, 0.309 mmol) in DMF (40 ml). The reaction was continued for 5 min and then 4-methoxybenzyl chloride (2.80 ml, 20.65 mmol) was slowly added. The reaction was continued at room temperature for 2 h and then at 40° C. for 20 h. The reaction was quenched with water. DCM was added and the phases separated. The organic phase was washed with HCl (1M, aq) and water, and dried over MgSO₄. Finally the product was purified by flash chromatography (SiO₂, heptane:ethyl acetate, product came at 5% ethyl acetate) to give the compound (1.41 g, 35%).

¹H NMR (400 MHz, CDCl₃) δ 7.28 (d, 2H), 6.85 (d, 2H), 4.36 (s, 2H), 3.77 (s, 3H), 3.74 (s, 3H), 1.48 (s, 6H).

### Step b: 2-[(4-methoxybenzyl)oxy]-2-methylpropan-1-ol

DIBAL-H (1.5 M in toluene, 17 ml) was added to methyl 2-[(4-methoxybenzyl)oxy]-2-methylpropanoate (1.52 g, 6.39 mmol) in toluene (10 ml) at −78° C. The reaction was continued at −78° C. for 5 min and then at room temperature for 1 h. The mixture was cooled to 0° C. Sodium tartrate (sat. aq, 50 ml) was added and the temperature increased to room temperature. The mixture was stirred for 3 h. The phases were separated and the water phase extracted with TBME. The combined organic phases were washed with water and dried over Na₂SO₄.

¹H NMR (400 MHz, CDCl₃) δ 7.28-7.21 (m, 2H), 6.90-6.84 (m, 2H), 4.37 (s, 2H), 3.79 (s, 3H), 3.46 (d, 2H) 2.09-2.01 (br, 1H), 1.25 (s, 6H).

### Step c: 1-[(1,1-dimethyl-2-phenoxyethoxy)methyl]-4-methoxybenzene

Diethyl azodicarboxylate (1.5 ml, 40% in toluene) was added to a mixture of 2-[(4-methoxybenzyl)oxy]-2-methylpropan-1-ol (1.00 g, 4.76 mmol), phenol (895 mg, 9.51 mmol) and triphenylphosphine (2.50 g, 9.51 mmol) in toluene (50 ml) at 0° C. The temperature was increased to 110° C. and the reaction continued for 4 h. The solvent was evaporated and the product was purified by flash chromatography (SiO₂, heptane:ethyl acetate, product came at 5% ethyl acetate) to give the compound (741 mg, 54%).

¹H NMR (400 MHz, CDCl₃) δ 7.41-7.35 (m, 4H), 7.08-7.02 (m, 3H), 7.00-6.94 (m. 2H), 4.60 (s, 2H), 4.00 (s, 2H), 3.84 (s, 3H), 1.51 (s, 6H).

### Step d: 2-methyl-1-phenoxypropan-2-ol

2,3-Dichloro-5,6-cyano-1,4-bezoquinone (365 g, 1.61 mmol) was added to a solution of 1-[(1,1-dimethyl-2-phenoxyethoxy)methyl]-4-methoxybenzene (46 mg, 0.16 mmol) in DCM (2 ml). The reaction was continued at room temperature for 24 h. The product was purified by flash chromatography (SiO₂, heptane:ethyl acetate, product came at 10% ethyl acetate) to give the compound (18 mg, 67%).

¹H NMR (400 MHz, CDCl₃) δ 7.34-7.22 (m, 2H), 7.00-6.88 (m, 3H), 3.79 (s, 2H), 2.26 (s, 1H), 1.34 (s, 6H).

### Step e: 1,1-dimethyl-2-phenoxyethyl bromoacetate

Prepared from bromoacetyl chloride (38 μl, 0.46 mmol) and 2-methyl-1-phenoxypropan-2-ol (64 mg, 0.39 mmol) as described in step b in Example 10 (123 mg, crude).

¹H NMR (400 MHz, CDCl₃) δ 7.30-7.20 (m, 2H), 6.98-6.87 (m, 3H), 4.09 (s, 2H), 3.74 (s, 2H), 1.57 (s, 6H).

### Step f: 1,1-dimethyl-2-phenoxyethyl 4-amino-1-(4-fluorophenyl)-2-methoxy-1H-imidazole-5-carboxylate

Prepared as described in step c in Example 10 (29 mg, 17% for two steps).

¹H NMR (400 MHz, CDCl₃) δ 7.30-7.20 (m, 2H), 7.20-7.14 (m, 2H), 7.04-6.96 (m, 2H), 6.96-6.89 (m, 1H), 6.86-6.76 (m, 2H), 5.20-5.06 (br, 2H), 3.91 (s, 3H), 3.86-3.71 (br, 2H), 1.41 (s, 6H).

MS m/z 400 (M+H)⁺.

### Step g: 1,1-dimethyl-2-phenoxyethyl 4-[2,4-dichlorobenzoyl)amino]-1-(4-fluorophenyl)-2-methoxy-1H-imidazole-5-carboxylate

Prepared as described in Example 2 (4 mg, 8%).

¹H NMR (400 MHz, CDCl₃) δ 9.75-9.60 (br, 1H), 7.65-7.55 (m, 1H), 7.43-7.35 (m, 1H), 7.34-7.30 (m, 1H), 7.24-7.15 (m, 2H), 7.06-6.99 (m, 2H), 6.99-6.90 (m, 2H), 6.83-6.77 (m, 1H), 6.73-6.67 (m, 2H), 4.20-3.90 (br, 3H), 3.73 (s, 2H), 1.37 (s, 6H).

HRMS Calcd for [C₂₈H₂₄Cl₂FN₃O₅+H]+: 572.116. Found: 572.114.

GTPγS(IC₅₀): 0.96≈1.0 μM

### Example 13

### 1-(ethoxycarbonyl)cyclopropyl 4-[(2,4-dichlorobenzoyl)amino]-1-(4-fluorophenyl)-2-methoxy-1H-imidazole-5-carboxylate

### Step a: Ethyl 1-(2-bromoacetoxy)cyclopropanecarboxylate

Prepared from bromoacetyl chloride (760 μl, 9.22 mmol) and ethyl 1-hydroxycyclopropanecarboxylate (1.0 g, 7.68 mmol) as described in step b in Example 10 (2.39, crude).

¹H NMR (400 MHz, CDCl₃) δ 4.17 (q, 2H), 3.82 (s, 2H), 1.27-1.18 (m, 7H).

### Step b: 1-(ethoxycarbonyl)cyclopropyl 4-amino-1-(4-fluorophenyl)-2-methoxy-1H-imidazole-5-carboxylate

Prepared as described in step c in Example 10 (353 mg, 29% for two steps).

¹H NMR (400 MHz, CDCl₃) δ 7.22-7.14 (m, 2H), 7.05-6.96 (m, 2H), 5.45-5.10 (br, 2H), 4.08 (q, 2H), 3.90 (s, 3H), 1.42-1.30 (br, 2H), 1.15 (t, 3H), 1.04-0.80 (br, 2H).

MS m/z 364 (M+H)⁺.

### Step c: 1-(ethoxycarbonyl)cyclopropyl 4-[(2,4-dichlorobenzoyl)amino]-1-(4-fluorophenyl)-2-methoxy-1H-imidazole-5-carboxylate

Prepared as described in Example 2 (80 mg, 52%).

¹H NMR (400 MHz, CDCl₃) δ 9.70-9.54 (br, 1H), 7.76-7.60 (m 1H), 7.45-7.41 (m, 1H), 7.35-7.30 (m, 1H), 7.25-7.19 (m, 2H),7.13-7.05 (m, 2H), 4.11 (q, 2H), 4.20-4.00 (br, 3H), 1.42-1.36 (m, 2H), 1.17 (t, 3H), 0.94-0.85 (m, 2H).

HRMS Calcd for [C₂₄H₂₀Cl₂FN₃O₆+H]+: 536.0.79, Found: 536.078.

GTPγS(IC₅₀): 2 μM

### Example 14

### 1-(5-ethyl-5-methyltetrahydrofuran-2-yl)-1-methylethyl 4-[(2,4-dichlorobenzoyl)amino]-1-(4-fluorophenyl)-2-methoxy-1H-imidazole-5-carboxylate

### Step a: 2-(5-ethyl-5-methyltetrahydrofuran-2-yl)propan-2-ol

A solution of 2-(5-methyl-5-vinyltetrahydrofuran-2-yl)propan-2-ol (0.05 M, 58.74 ml) in methanol was hydrogenated using H-cube™ (10% Pd/C cartridge, 30° C., full mode, 1 ml/min) (448 mg, 88%).

¹H NMR (400 MHz, CDCl₃) δ 3.72-3.58 (m, 1H), 2.34-2.12 (br, 1H), 1.80-1.60 (m, 3H), 1.60-1.48 (m, 1H), 1.48-1.37 (m, 2H), 1.10 (s, 3H), 1.07 (d, 3H), 1.02 (s, 3H), 0.08 (dt, 3H).

### Step b: 1-(5-ethyl-5-methyltetrahydrofuran-2-yl)-1-methylethyl bromoacetate

Prepared from bromoacetyl chloride (257 μl, 3.12 mmol) and 2-(5-ethyl-5-methyltetrahydrofuran-2-yl)propan-2-ol (448 mg, 2.6 mmol) as described in step b in Example 10 (777 mg, crude).

¹H NMR (400 MHz, CDCl₃) δ 4.10-3.95 (m, 1H), 3.74 (d, 2H), 2.00-1.49 (m, 6H), 1.47 (s, 3H), 1.45 (s, 3H), 1.16 (d, 3H), 0.87 (dt, 3H).

### Step c: 1-(5-ethyl-5-methyltetrahydrofuran-2-yl)-1-methylethyl 4-amino-1-(4-fluorophenyl)-2-methoxy-1H-imidazole-5-carboxylate

Prepared from 1-(5-ethyl-5-methyltetrahydrofuran-2-yl)-1-methylethyl bromoacetate (142 mg, 0.48 mmol) and methyl N′-cyano-N-(4-fluorophenyl)imidocarbamate (80 mg, 0.41 mmol) as described in step c in Example 10 (136 mg, crude).

MS m/z 406 (M+H)⁺.

### Step d: 1-(5-ethyl-5-methyltetrahydrofuran-2-yl)-1-methylethyl 4-[(2,4-dichlorobenzoyl) amino]-1-(4-fluorophenyl)-2-methoxy-1H-imidazole-5-carboxylate

Prepared as described in Example 2 (17 mg, 7% for three steps).

¹H NMR (400 MHz CDCl₃) δ 9.69 (d, 1H), 7.68-7.55 (m, 1H), 7.46-7.41 (m, 1H), 7.34-7.28 (m, 1H), 7.22-7.17 (m, 2H), 7.14-7.06 (m, 2H), 4.05-3.85 (m, 4H), 1.80-1.30 (m, 6H), 1.41 (s, 6H), 1.25 (dd, 3H), 0.80 (dt, 3H).

HRMS Calcd for [C₂₈H₃₀Cl₂FN₃O₅+H]+: 578.162. Found: 578.166.

GTPγS(IC₅₀): 2.1 μM

### Example 15

### 2-methoxy-1,1-dimethylethyl 1-(1,3-benzodioxol-5-yl)-4-[(2,3-dihydro-1,4-benzodioxin-2-ylcarbonyl)amino]-2-methoxy-1H-imidazole-5-carboxylate

### Step a: Methyl N-1,3-benzodioxol-5-yl-N′-cyanoimidothiocarbamate

Prepared as described in step a in Example 1 (2.23 g, 87%).

¹H-NMR (400 MHz, DMSO) δ 7.00-6.95 (m, 1H),6.91-6.84 (m, 1H), 6.83-6.78 (m, 1H), 6.01 (s, 2H), 2.60 (3H).

### Step b: 2-methoxy-1,1-dimethylethyl bromoacetare

Prepared as described in step b in Example 10 (5.73 g, 54%).

¹H-NMR (400 MHz, CDCl₃) δ 3.75 (s 2H) 3.49 (s, 2H), 3.35 (s, 3H), 1.44 (s, 6H).

### Step c: 2-Methoxy-1,1-dimethylethyl 4-amino-1-(1,3-benzodioxol-5-yl-2-methoxy-1H-imidazole-5-carboxylate

Prepared as described in step b in Example 1.

(176 mg, 12%)

¹H-NMR (400 MHz, CDCl₃) δ 6.80-6.74 (m, 1H), 6.71-6.65 (m, 2H), 5.96 (s, 2H), 5.14 (s, 2H), 3.91 (s, 3H), 3.36-3.25 (m, 5H), 1.33 (s, 6H).

### Step d: 2-methoxy-1,1-dimethylethyl 1-(1,3-benzodioxol-5-yl)-4-[(2,3-dihydro-1,4-benzodioxin-2-ylcarbonyl)amino]-2-methoxy-1H-imidazole-5-carboxylate

2-Methoxy-1,1-dimethylethyl 4-amino-1-(1,3-benzodioxol-5-yl)-2-methoxy-1H-imidazole-5-carboxylate (44.0 mg, 0.121 mmol) and triethylamine (34 μl, 0.242 mmol) were dissolved in DCM (2.5 ml). 2,3-Dihydro-1,4-benzodioxine-2-carbonyl chloride (48.1 mg, 0.242 mmol) was added dropwise. Subsequently the reaction was stirred overnight. Sodium bicarbonate (1 M, aq, 2 ml) was added and the mixture was poured on a phase separator. The product was rinsed through with DCM and the organic phase was collected and evaporated. Eventually the residue was purified with preparatory HPLC (37.3 mg, 58%).

¹H NMR (400 MHz, CDCl₃) δ 1.32 (s, 6H), 3.26 (s, 3H), 3.35 (s, 2H), 4.10 (s, 3H), 4.33 (m, 1H), 4.65 (dd, 1H), 4.85 (s, 1H), 6.04 (s, 2H), 6.70 (m, 2H) 6.85 (m, 1H), 6.92 (m, 3H), 7.11 (m, 1H), 10.12 (s, 1H).

MS m/z 526 (M+H)⁺.

GTPγS(IC₅₀): 1.4 μM

Examples 16 and 17 were prepared in an analogous method to Example 15.

### Example 16

### 2-methoxy-1,1-dimethylethyl 1-(1,3-benzodioxol-5-yl)-4-[(4-chlorobenzoyl)amino]-2-methoxy-1H-imidazole-5-carboxylate

(29.3 mg, 47%).

MS m/z 502, 504 (M+H)⁺.

GTP≡S(IC₅₀): 1.2 μM

### Example 17

### 2-methoxy-1,1-dimethylethyl 1-(1,3-benzodioxol-5-yl)-4-{[(benzyloxy)acetyl]amino}-2-methoxy-1H-imidazole-5-carboxylate

(37.0 mg. 58%).

MS m/z 512 (M+H)⁺.

GTPγS(IC₅₀): 3.4 μM

### Example 18

### 2-methoxy-1,1-dimethylethyl 4-[(2,4-dichlorobenzoyl)amino]-2-methoxy-1-(3-methoxyphenyl)-1H-imidazole-5-carboxylate

### Step a: methyl N′-cyano-N-(3-methoxyphenyl)imidothiocarbamate

Prepared as described in step a in Example 1.

(601 mg, 67%)

¹H-NMR (400 MHz, CD₃OD) δ 7.29-7.23 (m, 1H), 7.02-6.95 (m, 2H), 6.85-6.80 (m, 1H), 3.78 (s, 3H), 2.63 (s, 3H).

### Step b: 2-methoxy-1,1-dimethylethyl 4-amino-2-methoxy-1-(3-methoxyphenyl)-1H-imidazole-5-carboxylate

Prepared as described in step b in Example 1.

(29 mg, 12%)

MS m/z 350 (M+H)⁺

### Step c: 2-methoxy-1,1-dimethylethyl 4-[(2,4-dichlorobenzoyl)amino]-2-methoxy-1-(3-methoxyphenyl)-1H-imidazole-5-carboxylate

Prepared as described in Example 15 (11.8 mg, 27%)

¹H NMR (400 MHz, CDCl₃) δ 9.70 (s, 1H), 7.62 (s, 1H), 7.44 (s, 1H), 7.31 (t, 2H), 6.96-6.91 (m, 1H), 6.84-6.78 (m, 1H), 6.77-6.73 (m, 1H), 4.40 (s, 3H), 3.80 (s, 3H), 3.24-3.00 (m, 5H), 1.24 (s, 6H).

MS m/z 522, 524, 526 (M+H)⁺

GTPγS(IC₅₀): 1.3 μM

### Example 19

### Tert-butyl 4-[(4-azidobenzoyl)amino]-2-ethyl-1-(4-fluorophenyl)-1H-imidazole-5-carboxylate

### Step a: Ethyl propanimidoate hydrochloride

Hydrogen chloride gas was passed into propionitrile (30 g) in ethanol (250 ml) at 0° C. The reaction mixture was kept at 4° C. for 19 h. Then, solvent was evaporated to afford the product as a solid (62 g, 85%).

### Step b: Ethyl N-cyanopropanimidoate

Cyanamide (22.93 g, 0.546 mol) was added to ethyl propanimidoate hydrochloride (62 g, 0.455 mol) in water (300 ml) followed by potassium phosphate dibasic (110.8 g, 0.637 mol) at 0° C. The organic layer was separated and concentrated to afford the product as a liquid (34.8 g, 61%).

### Step c: N′-cyano-N-(4-fluorophenyl)propanimidamide

4-Fluoro aniline (30 g, 0.275 mol) was added to a stirred solution of ethyl N-cyanopropanimidoate (34.7 g, 0.275 mol) in ethanol (300 ml) at room temperature and refluxed for overnight. After completion of reaction the solvent was evaporated to afford crude product. Further purification was done through silica column chromatography (EtOAc/petroleum ether 1:9) to afford the product as a solid (34 g, 69%).

### Step d: Tert-butyl 4-amino-2-ethyl-1-(4-fluorophenyl-1H-imidazole-5-carboxylate

Step-4 and Step-5

Prepared as described in step c in Example 10 (5 g, 55%).

¹H-NMR (500 MHz, CD₃OD) δ 7.23-7.28 (m, 2H), 7.30-7.34 (m, 2H), 2.39 (q, 7.6 Hz, 2H), 1.23 (s, 9H), 1.13 (t, 7.6 Hz, 3H).

### Step e: 4-azidobenzoyl chloride

4-azidobenzoic acid (2.40 g, 14.74 mmol) was suspended in DCM (20 ml). Oxalyl chloride (1.5 ml, 17.73 mmol) was added and the reaction mixture was stirred at room temperature for 20 min. DMF (10 μl) was added and stirring continued for 1 h. The solvents were evaporated (2.60 g, 97%).

### Step f: Tert-butyl 4-[(4-azidobenzoyl)amino]-2-ethyl-1-(4-fluorophenyl)-1H-imidazole-5-carboxylate

Prepared as described in Example 2 (299 mg, 91%).

¹H-NMR (400 MHz, CDCl₃) δ 10.04 (s, 1H),7.98 (d, 8.7 Hz, 2H), 7.10-7.21 (m, 6H), 2.51 (q, 7.5 Hz, 2H), 1.15-1.19 (m, 12H).

HRMS Calcd for [C₂₃H₂₃FN₆O₃+H]⁺: 451.190. Found: 451.190.

GTPγS (IC₅₀): 1.1 μM

### Example 20

### Tert-butyl 4-[(2,4-dichlorobenzoyl)amino]-2-ethyl-1-(4-fluorophenyl)-1H-imidazole-5-carboxylate

Prepared as described in Example 2 from Tert-butyl 4-amino-2-ethyl-1-(4-fluorophenyl)-1H-imidazole-5-carboxylate of Ex. 19, step d (112 mg, 24%).

¹H-NMR (400 MHz, CDCl₃) δ 9.69 (br, 1H), 7.74-7.58 (m, 1H), 7.44 (s, 1H), 7.35-7.27 (m, 1H), 7.27-7.09 (m, 4H), 2.60-2.39 (ma 2H) 1.22-1.07 (m, 12H).

HRMS Calcd for [C₂₃H₂₂Cl₂FN₃O₃+H]⁺: 478.1 10. Found: 478.110.

GTPγS(IC₅₀): 3.7 μM

Analysis

LC-MS analysis was performed using a Micromass 8 probe MUX-LTC ESP+ system, purity being determined by single wavelength (254nm) UV detection. Chromatography was performed over an Xterra™ MS C8 3.5 um, 4.6×30 mm column, 8 in parallel. The flow of 15 ml/min was split over the 8 columns to give a flow rate of 1.9 ml/min. The 10-minute chromatography gradient was as follows:

Mobile Phase A: 95% ACN+5% 0.010 M NH₄OAc

Mobile Phase B: 5% ACN+95% 0.010 M NH₄OAc

| | | |
|---|---|---|
| 10 min | 0.0 min | 0% A |
| | 8.0 min | 100% A |
| | 9.0 min | 100% A |
| | 9.1 min | 0% A |

NMR analysis was performed at 400 MHz.

Biological Evaluation

Effects of the Positive Allosteric GABA_{B} Receptor Modulator in a Functional in vitro Assay.

The effect of GABA and baclofen on intracellular calcium release in CHO cells expressing the GABA_{B(1A,2)} receptor heterodimer was studied in the presence or absence of the positive allosteric modulator. The positive allosteric modulator according to the invention increased both the potency and the efficacy of GABA.

The potency of the compounds i.e. the ability of the compounds to reduce the EC₅₀ of GABA was revealed by the concentration required to reduce GABA's EC₅₀ by 50%. These potencies were similar to the potency reported for CGP7930 (can be purchased from Tocris, Northpoint, Fourth Way, Avonmouth, Bristol, BS11 8TA, UK) by Urwyler et al. CGP7930 increases the potency of GABA from EC₅₀ of about 170-180 nM to EC₅₀ of about 35-50 nM.

Experimental Procedures

Materials

Nut mix F-12 (Ham) cell culture media, OPTI-MEM I reduced serum medium, Fetal bovine serum (FBS), penicillin/streptomycin solution (PEST), geneticin, HEPES (4-(2-hydroxyethyl)-1-piperazineethanesulfonic acid (buffer), 1 M solution), Hank's Balanced Salt Solution (HBSS) and zeocin were from Life technologies (Paisley, Scotland); Polyethyleneimine, probenicid, baclofen and γ-aminobutyric acid (GABA) were from Sigma (St Louis, USA); Fluo-3 AM was from Molecular Probes (Oregon, USA). 4-Amino-n-[2,3-³H]butyric acid ([³H]GABA) was from Amersham Pharmacia Biotech (Uppsala, Sweden).

Generation of Cell Lines Expressing the GABA_{B} Receptor

GABA_{B}R1a and GABA_{B}R2 were cloned from human brain cDNA and subcloned into pCI-Neo (Promega) and pALTER-1(Promega), respectively. A GABA_{B}R1a-G_{αqi5} fusion protein expression vector was constructed using the pCI-Neo-GABA_{B}R1a cDNA plasmid and pLEC1-G_{αqi5} (Molecular Devices, Calif.). In order to make the G_{αqi5} pertussis toxin insensitive, Cys356 was mutated to Gly using standard PCR methodology with the primers 5′-GGATCCATGGCATGCTGCCTGAGCGA-3′ (forward) and 5′-GCGGCCG CTCAGAAGAGGCCGCCGTCCTT-3′ (reverse). The G_{αqi5mut} cDNA was ligated into the BamHI and NotI sites of pcDNA3.0 (Invitrogen). The GABA_{B} R1a coding sequence was amplified by PCR from pCI-Neo-GABA_{B}R1a using the primers, 5′-GGATCCCCGGGGAGCCGGGCCC-3′ (forward) and 5′-GGATCCCTTATAAAGCAAATGCACTCGA-3′ (reverse) and subcloned into the BamHI site of pcDNA3.0-G_{αqis5mut}.

In order to optimise the Kozak consensus sequence of GABA_{B}R2, in situ mutagenesis was performed using the Altered Sites Mutagenesis kit according to manufacturer's instruction (Promega) with the following primer, 5′-GAATTCGCACCATGGCTTCCC-3′. The optimised GABA_{B}R2 was then restricted from pALTER-1 with Xho I+Kpn I and subcloned into the mammalian expression vector pcDNA3.1(-)/Zeo (Invitrogen) to produce the final construct, pcDNA3.1(-)/Zeo-GABA_{B}R2.

For generation of stable cell lines, CHO-K1 cells were grown in Nut mix F-12 (Ham) media supplemented with 10% FBS, 100 U/ml Penicillin and 100 μg/ml Streptomycin at 37° C. in a humidified CO₂-incubator. The cells were detached with 1 mM EDTA in PBS and 1 million cells were seeded in 100 mm petri dishes. After 24 hours the culture media was replaced with OptiMEM and incubated for 1 hour in a CO₂-incubator.

For generation of a cell line expressing the GABA_{B}R1a/GABA_{B}R2 heterodimer, GABA_{B}R1a plasmid DNA (4 μg) GABA_{B}R2 plasmid DNA (4 μg) and lipofectamine (24 μl) were mixed in 5 ml OptiMEM and incubated for 45 minutes at room temperature. The cells were exposed to the transfection medium for 5 hours, which then was replaced with culture medium. The cells were cultured for an additional 10 days before selection agents (300 μg/ml hygromycin and 400 μg/ml geneticin) were added. Twenty-four days after transfection, single cell sorting into 96-well plates by flow cytometry was performed using a FACS Vantage SE (Becton Dickinson, Palo Alto, Calif.), After expansion, the GABA_{B} receptor functional response was tested using the FLIPR assay described below. The clone with the highest functional response was collected, expanded and then subcloned by single cell sorting. The clonal cell line with the highest peak response in the FLIPR was used in the present study.

For generation of a stable cell line expressing GABA_{B}R1a-G_{αqi5} fusion protein and GABA_{B}R2, GABA_{B}R1a-G_{αqi5mut} plasmid DNA (8 μg) GABA_{B}R2 plasmid DNA (8 μg) and lipofectamine (24 μl) were mixed in 5 ml OptiMEM and incubated for 45 minutes at room temperature. The cells were exposed to the transfection medium for 5 hours, which then was replaced with culture medium. After forty-eight hours, the cells were detached and seeded in 6 well plates (2000 cells well) and grown in culture medium supplemented with geneticin (400 μg/ml) and zeocin (250 μg/ml). After 4 days, cells from single colonies were collected and transferred to a 24-well plate. After 10 days, the cell clones were seeded in T-25 flasks and grown for another 16 days before they were tested for GABA_{B} receptor mediated functional response. The clones that showed the highest peak response were collected and subcloned by seeding the cells in 6-well plates (1000 cells/well) and repeating the steps described above. The clonal cell line that gave the highest peak response in the FLIPR was used in the present study.

Measurement of GABA_{B} Receptor Dependent Release of Intracellular Calcium in the FLIPR

Measurement of GABA_{B} receptor dependent release of intracellular calcium in the fluorescence imaging plate reader (FLIPR) was performed as described by Coward et al. Anal. Biochem. (1999) 270, 242-248, with some modifications. Transfected CHO cells were cultivated in Nut Mix F-12 (HAM) with Glutamax-I and supplemented with 10%, 100 U/ml penicillin and 100 μg/ml streptomycin, 250 μg/ml zeocin and 400 μg/ml geneticin. Twenty-four hours prior to the experiment the cells (35,000 cells/well) were seeded in black-walled 96-well poly-D-lysine coated plates (Becton Dickinson, Bedford, UK) in culture medium without selection agents. The cell culture medium was aspirated and 100 μl of Fluo-3 loading solution (4 μM Fluo-3, 2.5 mM probenecid and 20 mM Hepes in Nut Mix F-12 (Ham)) was added. After incubation for I hour at 37° C. in a 5% CO₂ incubator, the dye-solution was aspirated and the cells were washed 2 times with 150 μl of wash solution (2.5 mM probenecid and 20 mM Hepes in HBSS) followed by addition of 150 μl of wash solution. The cells were then assayed in a fluorescence imaging plate reader (Molecular Devices Corp., Calif., USA). Test compounds were diluted to 50 μM concentrations in HBSS containing 20 mM Hepes and 5% DMSO and added in a volume of 50 μl. The fluorescence was sampled every second for 60 s (10 s before and 50 s after the addition of test compound) before GABA (50 μl 7.6 nM-150 μM) was added and sampling continued every sixth second for additional 120 seconds.

GTPγS

[³⁵S]-GTPγS binding assays were performed at 30° C. for 45 min in membrane buffer (100 mM NaCl, 5 mM, 1 mM EDTA, 50 mM HEPES, pH 7.4) containing 0.025 μg/μl of membrane protein (prepared from the cell lines described above) with 0.01% bovine serum albumin (fatty acid free): 10 μM GDP, 100 μM DTT and 0.53 nM [³⁵S]-GTPγS (Amersham-Pharmacia Biotech) in a final volume of 200 μl. Non-specific binding was determined in the presence of 20 μM GTPγS. The reaction was started by the addition of GABA at concentration between 1 mM and 0.1 nM in the presence or absence of the required concentration of PAM. The reaction was terminated by addition of ice-cold wash buffer (50 mM Tris-HCl, 5 mM MgCl₂, 50 mM NaCl, pH 7.4) followed by rapid filtration under vacuum through Printed Filtermat A glass fiber filters (Wallac) (0.05% PEI treated) using a Micro 96 Harvester (Skatron Instruments). The filters were dried for 30 min at 50° C., then a paraffin scintillant pad was melted onto the filters and the bound radioactivity was determined using a 1450 Microbeta Trilux (Wallac) scintillation counter.

Calculations

GABA dose-response curves in the presence and absence of test compounds were constructed using the 4-parameter logistic equation, y=yₘₐₓ+((yₘᵢₙ−yₘₐₓ)/1+(x/C)^{D}), where C=EC₅₀ and D=slope factor.

The potency of PAM in GTPγS assays was determined by plotting the log EC₅₀ for GABA against the log concentration of the positive allosteric modulator in the presence of which the measurement was performed.

Generally, the potency of the compounds of the invention ranges from EC₅₀_{^{S}} between 20 μM and 0.001 μM. Examples of individual EC₅₀ values:

| Compound | EC₅₀ |
|---|---|
| Tert-butyl 4-[(2,4-dichlorobenzoyl)amino]-1- | 2.16 μM |
| (4-fluorophenyl)-2-methoxy-1H-imidazole-5-carboxylate | |
| 1,1-dimethyl-2-phenoxyethyl 4-[(2,4- | 0.96 μM |
| dichlorobenzoyl)amino]-1-(4-fluorophenyl)-2-methoxy- | |
| 1H-imidazole-5-carboxylate | |

Effect of Compounds in IBS Model (Colorectal Distension)

Colorectal Distension (CRD)

For CRD, a 3 cm polyethylene balloon with a connecting catheter (made in-house) was inserted in the distal colon, 2 cm from the base of the balloon to the anus, during light isoflurane anaesthesia (Forene®, Abbott Scandinavia AB, Sweden). The catheter was fixed to the base of the tail with tape. At the same time, an intravenous catheter (Neoflon®, Becton Dickinson AB, Sweden) was inserted in a tail vein for compounds administration. Thereafter, rats were placed in Bollman cages and allowed to recover from sedation for at least 15 min before starting the experiments.

During the CRD procedure, the balloons were connected to pressure transducers (P-602, CFM-k33, 100 mmHg; Bronkhorst Hi-Tec, Veenendal, The Netherlands). A customized barostat (AstraZeneca, Mölndal, Sweden) was used to control the air inflation and intraballoon pressure. A customized computer software (PharmLab on-line 4.0.1) running on a standard PC was used to control the barostat and to perform data collection and storage. The distension paradigm generated by the barostat were achieved by generating pulse patterns on an analog output channel. The CRD paradigms used consisted on repeated phasic distensions, 12 times at 80 mmHg, with a pulse duration of 30 s at 5 min intervals.

Responses to CRD were assessed by recording and quantitation of phasic changes in intraballoon pressure during the distending pulses. Pressue oscillations during the isobaric inflation of the intracolonic balloon reflect abdominal muscle contractions associated to the distension procedure and, therefore, are considered a valid assessment of the visceromotor response (VMR) associated to the presence of pain of visceral origin.

Data Collection and Analysis

The balloon pressure signals were sampled at 50 Hz and afterwards subjected to digital filtering. A highpass filter at 1 Hz was used to separate the contraction-induced pressure changes from the slow varying pressure generated by the barostat. A resistance in the airflow between the pressure generator and the pressure transducer further enhanced the pressure variations induced by abdominal contractions of the animal. In addition, a band-stop filtere at 49-51 Hz was used to remove line frequency interference. A customized computer software (PharmLab off-line 4.0.1) was used to quantify the phasic changes of the balloon pressure signals. The average rectified value (ARV) of the balloon pressure signals was calculated for the 30 s period before the pulse (baseline activity) and for the duration of the pulse (as a measure of the VMR to distension). When performing pulses analysis, the first and last second of each pulse were excluded since they reflect artefact signals produced by the barostat during inflation and deflation of the balloon and do not originate from the animal.

1. A compound which is selected from one or more of the following:
Tert-butyl 4-({[3-chloro-4-(isopropylsulfonyl)-2-thienyl]carbonyl}amino)-1-(4-fluorophenyl)-2-methoxy-1H-imidazole-5-carboxylate;
Tert-butyl 4-[(4-chlorobenzoyl)amino]-1-(4-fluorophenyl)-2-methoxy-1H-imidazole-5-carboxylate;
Tert-butyl 4-[(2,3-dihydro-1,4-benzodioxin-2-ylcarbonyl)amino]-1-(4-fluorophenyl)-2-methoxy-1H-imidazole-5-carboxylate;
Tert-butyl 4-({[1-(4-chlorophenyl)cyclopentyl]carbonyl}amino)-1-(4-fluorophenyl)-2-methoxy-1H-imidazole-5-carboxylate;
Tert-butyl 1-(4-fluorophenyl)-2-methoxy-4-[(3,3,3-trifluoro-2-methoxy-2-phenylpropanoyl)amino]-1H-imidazole-5-carboxylate;
Tert-butyl 4-({[1-(4-chlorophenyl)-5-(trifluoromethyl)-1H-pyrazol-4-yl]carbonyl}amino)-1-(4-fluorophenyl)-2-methoxy-1H-imidazole-5-carboxylate;
Tert-butyl 1-(4-fluorophenyl)-2-methoxy-4-{[(6-phenoxypyridin-3-yl)carbonyl]amino}-1H-imidazole-5-carboxylate;
Tert-butyl 1-(4-fluorophenyl)-2-methoxy-4-[(2,4,6-trifluorobenzoyl)amino]-1H-imidazole-5-carboxylate;
Tert-butyl 4-[(2,4-dichlorobenzoyl)amino]-1-(4-fluorophenyl)-2-methoxy-1H-imidazole-5-carboxylate;
2-(2-methoxyethoxy)-1,1-dimethylethyl 4-[(2,4-dichlorobenzoyl)amino]-1-(4-fluorophenyl)-2-methoxy-1H-imidazole-5-carboxylate;
1,1-dimethyl-2-(2,2,2-trifluoroethoxy)ethyl 4-[(2,4-dichlorobenzoyl)amino]-1-(4-fluorophenyl)-2-methoxy-1H-imidazole-5-carboxylate;
1,1-dimethyl-2-phenoxyethyl 4-[(2,4-dichlorobenzoyl)amino]-1-(4-fluorophenyl)-2-methoxy-1H-imidazole-5-carboxylate;
1-(ethoxycarbonyl)cyclopropyl 4-[(2,4-dichlorobenzoyl)amino]-1-(4-fluorophenyl)-2-methoxy-1H-imidazole-5-carboxylate;
1-(5-ethyl-5-methyltetrahydrofuran-2-yl)-1-methylethyl 4-[(2,4-dichlorobenzoyl)amino]-1-(4-fluorophenyl)-2-methoxy-1H-imidazole-5-carboxylate;
2-methoxy-1,1-dimethylethyl 1-(1,3-benzodioxol-5-yl)-4-[(2,3-dihydro-1,4-benzodioxin-2-ylcarbonyl)amino]-2-methoxy-1H-imidazole-5-carboxylate;
2-methoxy-1,1-dimethylethyl 1-(1,3-benzodioxol-5-yl)-4-[(4-chlorobenzoyl)amino]-2-methoxy-1H-imidazole-5-carboxylate;
2-methoxy-1,1-dimethylethyl 1-(1,3-benzodioxol-5-yl)-4-{[(benzyloxy)acetyl]amino}-2-methoxy-1H-imidazole-5-carboxylate;
2-methoxy-1,1-dimethylethyl 4-[(2,4-dichlorobenzoyl)amino]-2-methoxy-1-(3-methoxyphenyl)-1H-imidazole-5-carboxylate;
Tert-butyl 4-[(4-azidobenzoyl)amino]-2-ethyl-1-(4-fluorophenyl)-1H-imidazole-5-carboxylate; and
Tert-butyl 4-[(2,4-dichlorobenzoyl)amino]-2-ethyl-1-(4-fluorophenyl)-1H-imidazole-5-carboxylate;
or a pharmaceutically acceptable salt thereof.

2-20. (canceled)

21. A pharmaceutical composition comprising a compound according to claim 1, or a pharmaceutically acceptable salt thereof, as an active ingredient and a pharmaceutically acceptable carrier or diluent.

22. A method for the treatment of gastroesophageal reflux disease (GERD) comprising administering an effective amount of a compound according to claim 1, optionally in combination with a GABA_{B} receptor agonist, to a subject.

23. A method for the prevention of reflux comprising administering an effective amount of a compound according to claim 1, optionally in combination with a GABA_{B} receptor agonist, to a subject.

24. A method for the inhibition of transient lower esophageal sphincter relaxations (TLESRs) comprising administering an effective amount of a compound according to claim 1, optionally in combination with a GABA_{B} receptor agonist, to a subject.

25. A method for the treatment of a functional gastrointestinal disorder comprising administering an effective amount of a compound according to claim 1, optionally in combination with a GABA_{B} receptor agonist, to a subject.

26. A method according to claim 25 wherein the functional gastrointestinal disorder is functional dyspepsia.

27. A method for the treatment of irritable bowel syndrome (IBS) comprising administering an effective amount of a compound according to claim 1, optionally in combination with a GABA_{B} receptor agonist, to a subject.

28. A method according to claim 27 wherein the IBS is constipation predominant IBS.

29. A method according to claim 27 wherein the IBS is diarrhea predominant IBS.

30. A method according to claim 27 wherein the IBS is alternating bowel movement predominant IBS.
